(19) United States
(12) Patent Application Publication
(10) Pub. No.: US 2024/0312604 A1
(43) Pub. Date: Sep. 19, 2024

(54) TUMOR CELL CONTENT EVALUATION METHOD, SYSTEM AND COMPUTER DEVICE

(71) Applicant: GUANGZHOU KINGMED CENTER FOR CLINICAL LABORATORY, Guangdong (CN)

(72) Inventors: Shuanlong CHE, Guangdong (CN); Pifu LUO, Guangdong (CN); Yinghua LI, Guangdong (CN); Si LIU, Guangdong (CN); Weisong QIU, Guangdong (CN); Tao WU, Guangdong (CN); Zhengpeng HA, Guangdong (CN); Haihui LV, Guangdong (CN)

(73) Assignee: GUANGZHOU KINGMED CENTER FOR CLINICAL LABORATORY, Guangdong (CN)

(21) Appl. No.: 18/280,242

(22) PCT Filed: Dec. 17, 2020
(86) PCT No.: PCT/CN2020/137029

§ 371 Date: Sep. 4, 2023

(30) Foreign Application Priority Data
Jul. 7, 2020 (CN) 202010644331.4

(51) Int. Cl. G16H 30/40

(52) U.S. Cl. CPC G16H 30/40

(57) ABSTRACT

Embodiments of this application disclose a tumor cell content evaluation method. A digital pathology slide image is obtained, and an effective pathological region is determined based on the digital pathology slide image; a tumor cell region corresponding to the effective pathological region is identified by using a deep learning-based pathology image classifier; and tumor cell content of the digital pathology slide image is determined based on the tumor cell region according to a preset evaluation rule. In this way, the tumor cell content of the digital pathology slide image is automatically evaluated, and accuracy and objectivity of evaluating the tumor cell content are improved. In addition, a tumor cell content evaluation system, a computer device, and a storage medium are further provided.

This application is based on and claims priority to Chinese Invention Patent Application No. 202010644331.4, filed on Jul. 7, 2020 and entitled “TUMOR CELL CONTENT EVALUATION METHOD AND SYSTEM, COMPUTER DEVICE, AND STORAGE MEDIUM”.

### TECHNICAL FIELD

This application relates to the field of image processing technologies, and in particular, to a tumor cell content evaluation method and system, a computer device, and a storage medium.

### BACKGROUND ART

Currently, the medical market has a large demand for gene detection and protein detection of tumor molecular pathology, and there are related molecular pathological examination solutions for common lung cancer, breast cancer, bowel cancer, and the like.

### SUMMARY OF THE INVENTION

### Technical Problem

Currently, a tumor patient that needs precise treatment such as targeted treatment and immunological treatment needs to provide a tumor tissue sample for molecular pathological examination. However, a pathological sample obtained through clinical surgical biopsy is affected by a plurality of factors such as a patient factor, a tumor factor, and biopsy sampling performed by a clinical surgeon. Consequently, tissue content and tumor content of each pathological sample sent for examination fluctuate in a wide range. For a sample with small tissue content or small tumor content, a molecular pathological examination technology platform often cannot detect the tissue content or the tumor content, resulting in a false-negative phenomenon; or two samples of a patient are sent for molecular pathological examination, but examination results are seriously inconsistent.

### Technical Solution of the Problem

Technical Solution Based on this, for the foregoing problems, it is necessary to provide a tumor cell content evaluation method and system, a computer device, and a storage medium, so as to improve objectivity and accuracy of evaluating tumor cell content.

A tumor cell content evaluation method is provided. The method includes:
obtaining a digital pathology slide image, and determining an effective pathological region based on the digital pathology slide image;
identifying a tumor cell region corresponding to the effective pathological region by using a deep learning-based pathology image classifier; and
determining tumor cell content of the digital pathology slide image based on the tumor cell region according to a preset evaluation rule.

A tumor cell content evaluation system is provided. The system includes:
a region determining module, configured to obtain a digital pathology slide image, and determine an effective pathological region based on the digital pathology slide image;
a tumor cell identification module, configured to identify a tumor cell region corresponding to the effective pathological region by using a deep learning-based pathology image classifier; and
a content evaluation module, configured to determine tumor cell content of the digital pathology slide image based on the tumor cell region according to a preset evaluation rule.

A computer device is provided, including a memory and a processor. The memory stores computer-readable instructions, and when the computer-readable instructions are executed by the processor, the processor is enabled to perform the following steps:
obtaining a digital pathology slide image, and determining an effective pathological region based on the digital pathology slide image;
identifying a tumor cell region corresponding to the effective pathological region by using a deep learning-based pathology image classifier; and
determining tumor cell content of the digital pathology slide image based on the tumor cell region according to a preset evaluation rule.

One or more non-volatile readable storage media storing computer-readable instructions are provided, where when the computer-readable instructions are executed by one or more processors, the one or more processors are enabled to perform the following steps:
obtaining a digital pathology slide image, and determining an effective pathological region based on the digital pathology slide image;
identifying a tumor cell region corresponding to the effective pathological region by using a deep learning-based pathology image classifier; and
determining tumor cell content of the digital pathology slide image based on the tumor cell region according to a preset evaluation rule.

### Advantageous Effect of the Invention

### Advantageous Effect

According to the foregoing tumor cell content evaluation method and system, computer device, and storage medium, the digital pathology slide image is obtained, and the effective pathological region is determined based on the digital pathology slide image; the tumor cell region corresponding to the effective pathological region is identified by using the deep learning-based pathology image classifier; and the tumor cell content of the digital pathology slide image is determined based on the tumor cell region according to the preset evaluation rule. In this way, tumor cell identification is performed on a pathology slide image by using a deep learning method, an identified cell region is comprehensively evaluated in a plurality of evaluation manners; and accuracy and objectivity of tumor cell identification are ensured by using a machine vision identification technology. Therefore, accuracy of evaluating the tumor cell content is further improved through comprehensive evaluation in the plurality of evaluation manners.

### BRIEF DESCRIPTION OF DRAWINGS

### Description of the Drawings

To describe the technical solutions in the embodiments of this application or in the conventional technology more clearly, the following briefly describes the accompanying drawings required for descriptions in the embodiments or the conventional technology. Apparently, the accompanying drawings in the following descriptions show merely some embodiments of this application, and a person of ordinary skill in the art may still derive other accompanying drawings from these accompanying drawings without creative efforts.

Wherein:

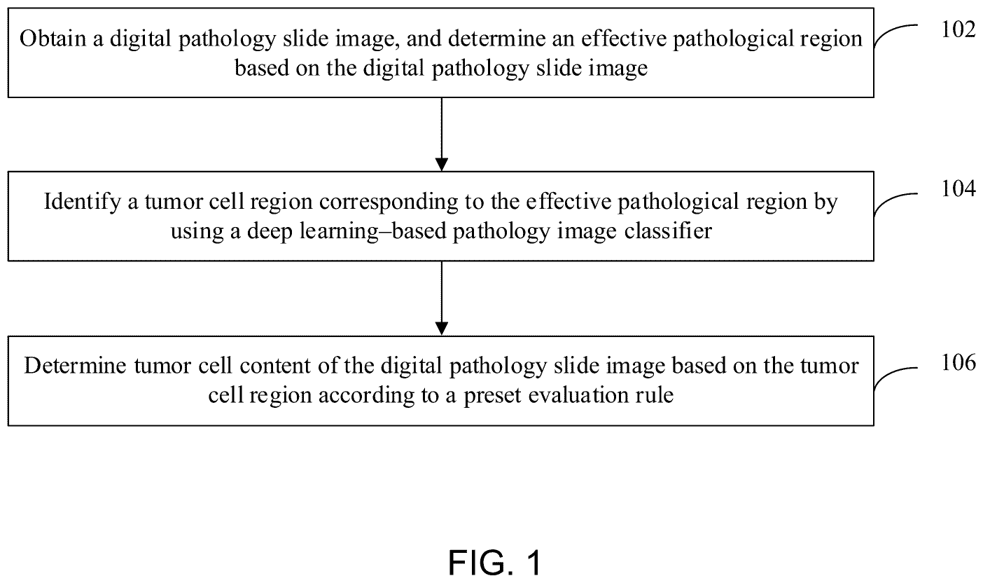

FIG. 1 is a flowchart of a tumor cell content evaluation method according to an embodiment;

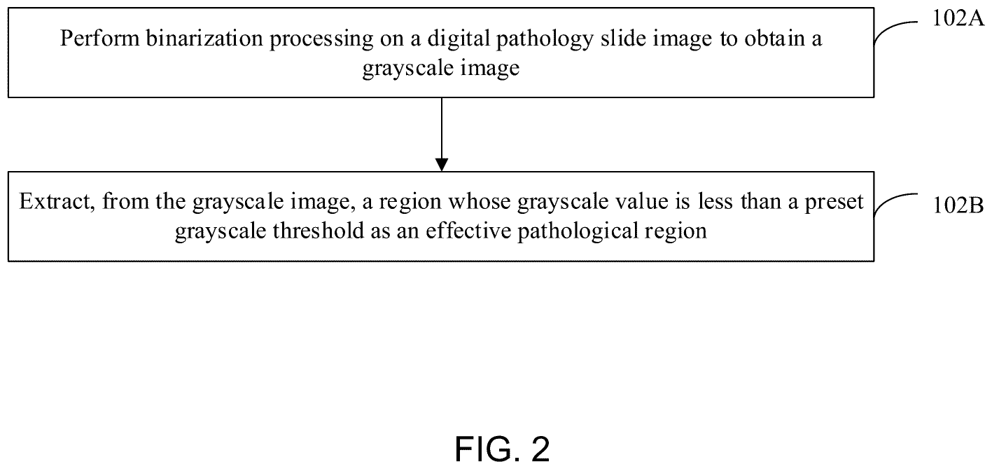

FIG. 2 is a flowchart of an effective pathological region determining method according to an embodiment;

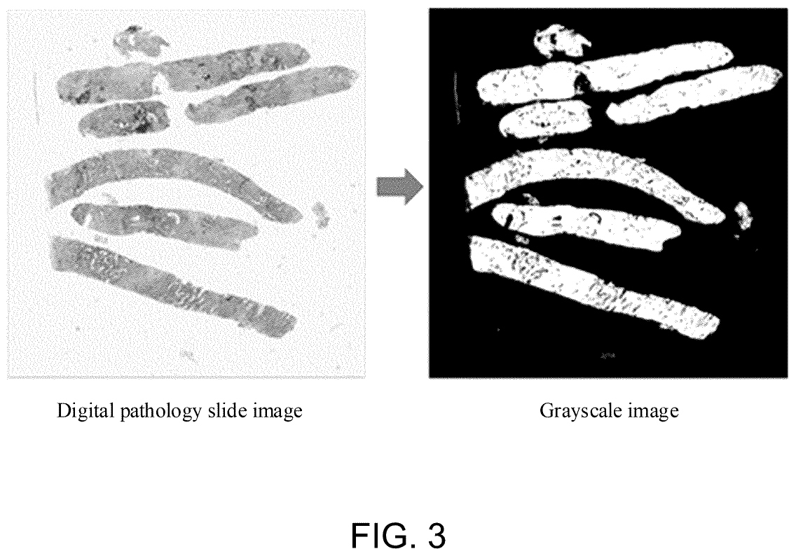

FIG. 3 is a diagram of comparison between a digital pathology slide image and a grayscale image according to an embodiment;

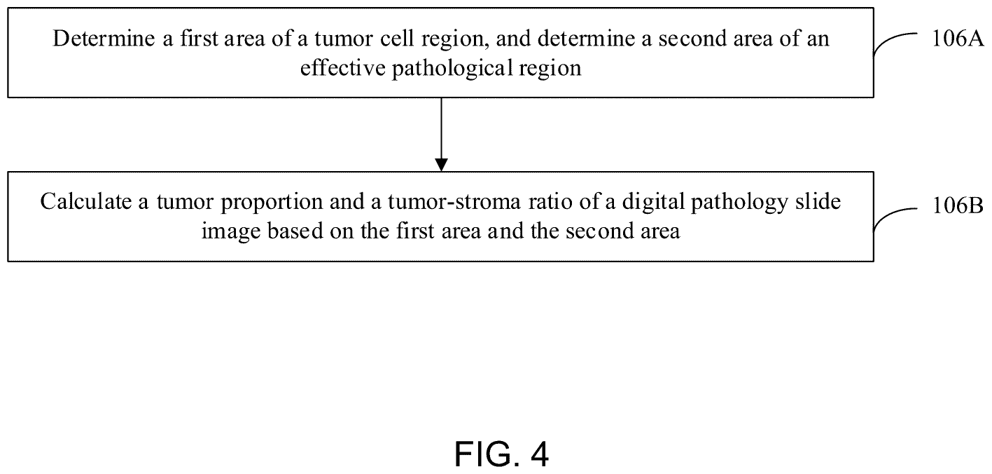

FIG. 4 is a flowchart of a tumor cell content evaluation method according to another embodiment;

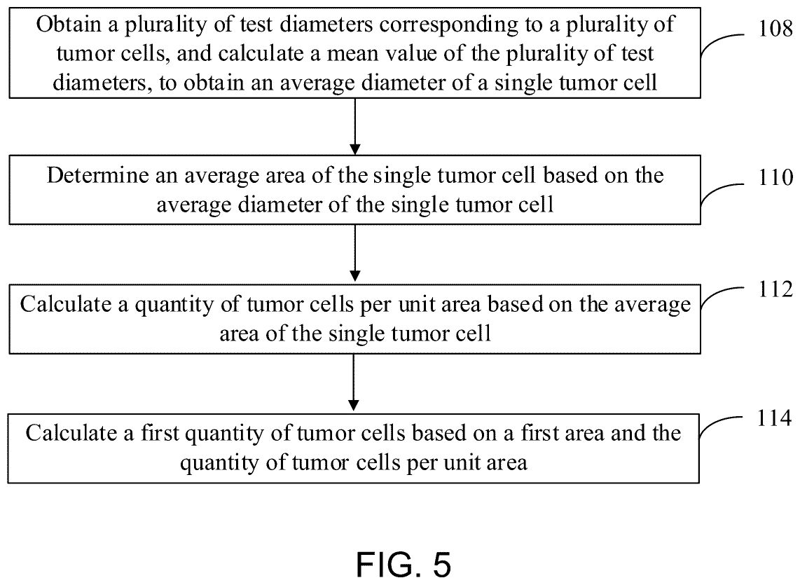

FIG. 5 is a flowchart of a tumor cell content evaluation method according to still another embodiment;

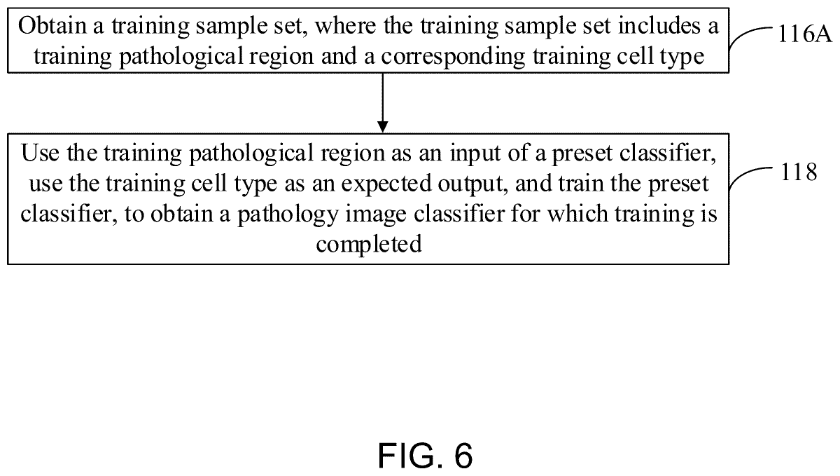

FIG. 6 is a flowchart of a tumor cell content evaluation method according to yet another embodiment;

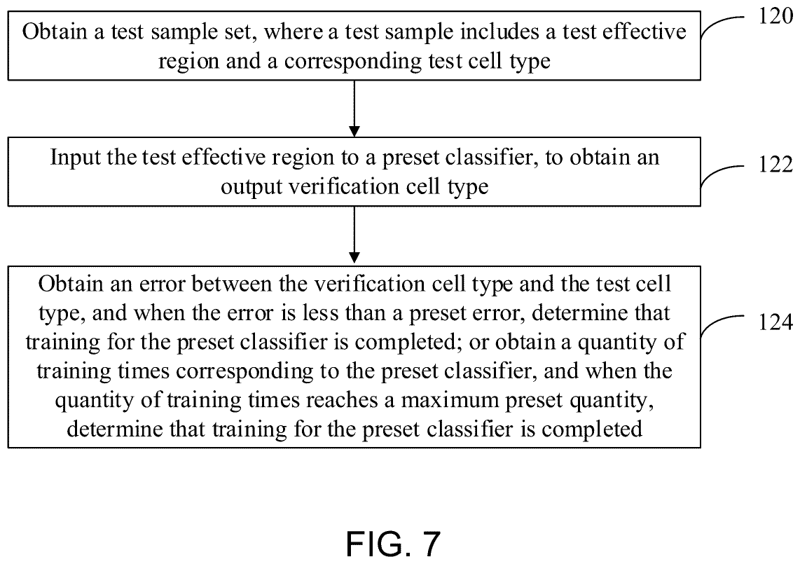

FIG. 7 is a flowchart of a tumor cell content evaluation method according to further another embodiment;

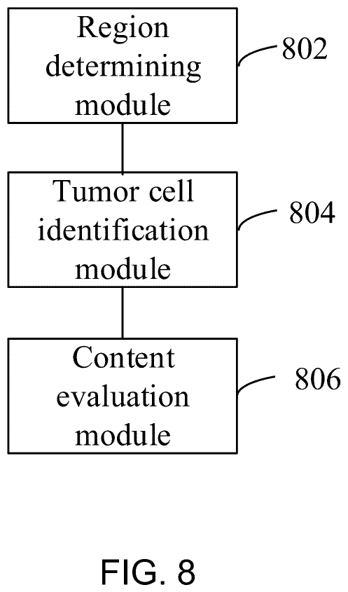

FIG. 8 is a block diagram of a structure of a tumor cell content evaluation system according to an embodiment; and

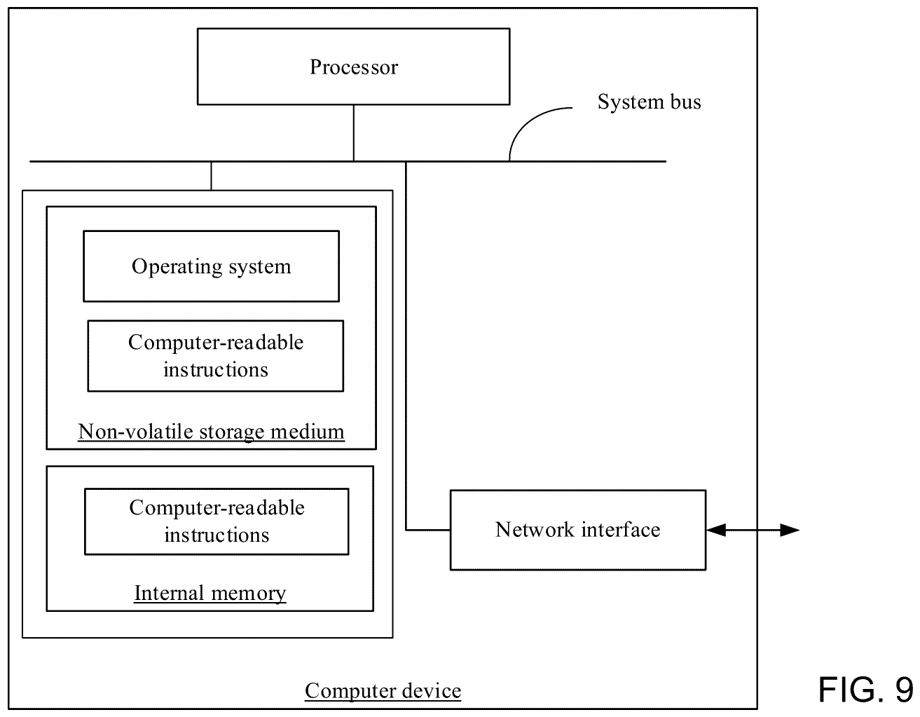

FIG. 9 is a block diagram of a structure of a computer device according to an embodiment.

### DETAILED DESCRIPTION OF THE EMBODIMENTS

### Embodiments of the Present Invention

The following clearly and completely describes the technical solutions in the embodiments of this application with reference to the accompanying drawings in the embodiments of this application. Apparently, the described embodiments are merely some rather than all of the embodiments of this application. All other embodiments obtained by a person of ordinary skill in the art based on the embodiments of this application without creative efforts shall fall within the protection scope of this application.

As shown in FIG. 1, in an embodiment, a tumor cell content evaluation method is provided. The tumor cell content evaluation method may be applied to a terminal or a server. In this embodiment, an example in which the tumor cell content evaluation method is applied to a server is used for description. The tumor cell content evaluation method specifically includes the following steps:
Step 102: Obtain a digital pathology slide image, and determine an effective pathological region based on the digital pathology slide image.

The digital pathology slide image is a pathology slide image obtained by displaying and saving a pathology slide in a manner of a digital image. Specifically, the digital pathology slide image may be obtained by performing digital scanning on a pathology slide of a biopsy sample by using a digital pathology scanner. The effective pathological region is a region including cells in the pathology slide, where the cells include normal and/or diseased cells. The digital pathology slide image is segmented to determine the effective pathological region, where an image segmentation method includes but is not limited to an image binarization processing method, a machine learning-based image segmentation model, or an edge detection algorithm. As a preference of this embodiment, a binarization processing manner is used. To be specific, a feature that average grayscale values of a cell region and a non-cell region are inconsistent is used, and a reasonable grayscale threshold is set, to filter out a part of the non-cell region to extract the effective pathological region. Compared with the machine learning-based image segmentation model or the edge detection algorithm, this method is simpler and faster.
Step 104: Identify a tumor cell region corresponding to the effective pathological region by using a deep learning-based pathology image classifier.

Deep learning is a method for performing representation learning on data in machine learning. An observed value (for example, an image) may be represented in a plurality of manners, for example, represented as a vector of each pixel intensity value or more abstractly represented as a series of edges or a region of a particular shape. It is easier to learn a task (for example, face recognition) from an instance by using a specific representation method. Deep learning is used to establish and simulate a neural network of a human brain for analyzing and learning. Deep learning is used to imitate a mechanism of the human brain to interpret data, for example, an image, a sound, and a text, and uses unsupervised or semi-supervised feature learning and an efficient algorithm based on hierarchical feature extraction to replace manual feature obtaining, which can help improve objectivity and accuracy of a prediction result. The pathology image classifier is a classifier, and can learn, by using a sample, a machine learning-based algorithm model having a classification capability. The pathology image classifier of this embodiment is configured to classify different effective pathological regions into a tumor cell region and a non-tumor cell region. Specifically, the pathology image classifier is a classifier that can use at least one machine learning-based model for classification. The machine learning-based model may be one or more of the following: a neural network (for example, a convolutional neural network or a BP neural network), a logical regression model, a support vector machine, a decision tree, a random forest, a perceptron, and another machine learning-based model. As a part of training of such a machine learning-based model, training inputs are images corresponding to various effective pathological regions. In this way, a relationship classifier corresponding to the effective pathological region and the tumor cell region or the non-tumor cell region is established through training. Therefore, the pathology image classifier has a capability of determining whether an input effective pathological region corresponds to the tumor cell region or the non-tumor cell region. In this embodiment, the pathology image classifier is a binary classifier, that is, two classification results are obtained, namely, the tumor cell region or the non-tumor cell region.

The non-tumor cell region is a region in which normal cells are located in the effective pathological regions, and the tumor cell region is a region in which diseased cells are located in the effective pathological regions, and is used by a doctor to analyze a cell content of the region to implement disease diagnosis. It may be understood that the tumor cell region in the effective pathological regions is identified in a deep learning manner, thereby automatically detecting the tumor cell region, and improving accuracy and objectivity of tumor cell identification.
Step 106: Determine tumor cell content of the digital pathology slide image based on the tumor cell region according to a preset evaluation rule.

The preset evaluation rule refers to a preset evaluation manner or indicator used to evaluate the tumor cell content, for example, evaluation may be performed based on an area of the tumor cell region, a quantity of cells, a tumor proportion, a tumor-stroma ratio, and the like. It may be understood that evaluation is performed according to the preset evaluation rule, thereby automatically evaluating the tumor cell content of the digital pathology slide image from a plurality of perspectives, ensuring comprehensiveness and objectivity of evaluation, improving accuracy and consistency of evaluating the tumor cell content, and avoiding failing to obtain a uniform and precise evaluation result due to a subjective deviation in manual analysis.

According to the tumor cell content evaluation method, the digital pathology slide image is obtained, and the effective pathological region is determined based on the digital pathology slide image; the tumor cell region corresponding to the effective pathological region is identified by using the deep learning-based pathology image classifier; and the tumor cell content of the digital pathology slide image is determined based on the tumor cell region according to the preset evaluation rule. In this way, the tumor cell content of the digital pathology slide image is automatically evaluated, and accuracy and objectivity of evaluating the tumor cell content are improved.

As shown in FIG. 2, in an embodiment, the determining an effective pathological region based on the digital pathology slide image includes:
Step 102A: Perform binarization processing on the digital pathology slide image to obtain a grayscale image.
Step 102B: Extract, from the grayscale image, a region whose grayscale value is less than a preset grayscale threshold as the effective pathological region.

In this embodiment, the effective pathological region is a region dyed by a red dyeing reagent and a blue dyeing reagent, a grayscale of a background region is colorless or white. A grayscale value of the background region is greater than a grayscale value of the effective pathological region. The preset grayscale threshold is determined based on a grayscale of the effective pathological region. Therefore, binarization processing is performed on the digital pathology slide image, so that a color picture is converted into a grayscale image, where the binarization processing includes but is not limited to global binarization, a histogram-based optimal threshold method, or a clustering-based OTSU threshold method. A digital pathology slide image of a lung cancer is used as an example. FIG. 3 is a diagram of comparison between a digital pathology slide image and a grayscale image. The region whose grayscale value is less than the preset grayscale threshold is extracted from the grayscale image, so that the effective pathological region is extracted, thereby facilitating further processing of the effective pathological region subsequently.

As shown in FIG. 4, in an embodiment, the determining tumor cell content of the digital pathology slide image based on the tumor cell region according to a preset evaluation rule includes:
Step 106A: Determine a first area of the tumor cell region, and determine a second area of the effective pathological region.
Step 106B: Calculate a tumor proportion and a tumor-stroma ratio of the digital pathology slide image based on the first area and the second area.

Specifically, the first area may be calculated based on the tumor cell region, and the second area may be calculated based on the effective pathological region. The tumor proportion and the tumor-stroma ratio are respectively calculated by using the following two formulas:

$$P 1 = S 1 / S 2 \times 100 \% ;$$

$$and$$

$$P 2 = S 1 / [ S 2 - S 1 ] \times 100 \% .$$

In the formulas, S1 represents the first area, S2 represents the second area, P1 represents the tumor proportion, and P2 represents the tumor-stroma ratio. In this embodiment, the tumor proportion and the tumor-stroma ratio are used as indicators for evaluating the tumor cell content, so that the doctor performs disease diagnosis based on the tumor proportion and the tumor-stroma ratio.

As shown in FIG. 5, in an embodiment, after the determining tumor cell content of the digital pathology slide image based on the tumor cell region according to a preset evaluation rule, the method further includes:
Step 108: Obtain a plurality of test diameters corresponding to a plurality of tumor cells, and calculate a mean value of the plurality of test diameters, to obtain an average diameter of a single tumor cell.
Step 110: Determine an average area of the single tumor cell based on the average diameter of the single tumor cell.
Step 112: Calculate a quantity of tumor cells per unit area based on the average area of the single tumor cell.
Step 114: Calculate a first quantity of tumor cells based on the first area and the quantity of tumor cells per unit area.

Specifically, a diameter of a tumor cell (for example, a lung cancer cell) is pre-tested. For example, the plurality of tumor cells (for example, 100 tumor cells) are tested to obtain 100 test diameters, and the mean value of the plurality of test diameters is calculated to obtain the average diameter of the single tumor cell. The average area of the single tumor cell is calculated based on the average diameter of the single tumor cell: S=π×(d/2)². d represents the average diameter, and S represents the average area. The quantity of tumor cells per unit area is calculated based on the average area of the single tumor cell, and the first area is multiplied by the quantity of tumor cells per unit area, to obtain the first quantity of tumor cells. In this embodiment, the first quantity of tumor cells in the tumor cell region is calculated, so that the doctor performs disease diagnosis and analysis based on the first quantity.

In an embodiment, after the determining tumor cell content of the digital pathology slide image based on the tumor cell region according to a preset evaluation rule, the method further includes:
performing cell segmentation on the tumor cell region by using a cell segmentation algorithm, to determine a second quantity of tumor cells.

The cell segmentation algorithm is a method used for cell segmentation, and includes but is not limited to a cell segmentation algorithm of an FCN full convolution neural network, a cell segmentation algorithm of a U-Net model, or a deep learning-based segmentation algorithm. Cells in the tumor cell region are segmented, and a quantity of cells is counted to obtain the second quantity, so that the doctor performs disease diagnosis and analysis based on the second quantity.

It should be noted that, in this embodiment, the second quantity is calculated based on a feature of the cell, and is more precise than the first quantity in step 114. Therefore, the second quantity is applicable to more examinations or disease diagnosis occasions.

As shown in FIG. 6, in an embodiment, the method further includes:
Step 116: Obtain a training sample set, where the training sample set includes a training pathological region and a corresponding training cell type.
Step 118: Use the training pathological region as an input of a preset classifier, use the training cell type as an expected output, and train the preset classifier, to obtain the pathology image classifier for which training is completed.

Specifically, samples of the tumor cell region and the non-tumor cell region that are labeled by the doctor are obtained, the training pathological region is used as the input of the preset classifier, the training cell type is used as the expected output, and the preset classifier is trained, to generate a cell type corresponding to the training pathological region in the training sample set, so as to train the preset classifier based on the expected output corresponding to the current training pathological region, to obtain the pathology image classifier for which training is completed.

In this embodiment, the training sample set includes the tumor cell region and the non-tumor cell region, thereby ensuring comprehensiveness of the training sample set. A more comprehensive and accurate cell type classification rule can be learned based on the cell type trained by using such a training sample set, thereby improving efficiency of training a preset machine learning-based classifier, and further improving efficiency of identifying the tumor cell region.

As shown in FIG. 7, in an embodiment, before the using the training pathological region as an input of a preset classifier, using the training cell type as an expected output, and training the preset classifier, to obtain the pathology image classifier for which training is completed, the method further includes:
Step 120: Obtain a test sample set, where the test sample set includes a test effective region and a corresponding test cell type.
Step 122: Input the test effective region to the preset classifier, to obtain an output verification cell type.
Step 124: Obtain an error between the verification cell type and the test cell type, and when the error is less than a preset error, determine that training for the preset classifier is completed; or
obtain a quantity of training times corresponding to the preset classifier, and when the quantity of training times reaches a maximum preset quantity, determine that training for the preset classifier is completed.

Specifically, the test sample set is obtained, and the test sample set includes the test effective region and the corresponding test cell type. The test sample set is predicted by using the trained machine learning-based classifier, and a known classification result of the test sample set is obtained, that is, an expected cell type is obtained. A prediction result obtained through prediction is compared with the known classification result, to obtain a corresponding classification prediction correct rate of the machine learning-based classifier. The error between the verification cell type and the expected cell type is obtained, and when the error is less than the preset error, it is determined that training for the preset classifier is completed; or the quantity of training times corresponding to the preset classifier is obtained, and when the quantity of training times reaches the maximum preset quantity, it is determined that training for the preset classifier is completed. Then, a parameter value used by the machine learning-based classifier is obtained. Otherwise, training for the machine learning-based classifier is continued by using the obtained parameter value and the test sample set.

In this embodiment, the parameter value is roughly positioned by using the test sample set, and a most appropriate parameter value may be found as much as possible by obtaining the error between the verification cell type and the expected cell type or obtaining the quantity of training times, to perform training by using the parameter value and the test sample set. In this way, the trained machine learning-based classifier can achieve higher accuracy in cell type distinguishing.

As shown in FIG. 8, in an embodiment, a tumor cell content evaluation system is provided. The system includes:
a region determining module 802, configured to obtain a digital pathology slide image, and determine an effective pathological region based on the digital pathology slide image;
a tumor cell identification module 804, configured to identify a tumor cell region corresponding to the effective pathological region by using a deep learning-based pathology image classifier; and
a content evaluation module 806, configured to determine tumor cell content of the digital pathology slide image based on the tumor cell region according to a preset evaluation rule.

In an embodiment, the region determining module includes:
a binarization processing unit, configured to perform binarization processing on the digital pathology slide image to obtain a grayscale image; and
a region determining unit, configured to extract, from the grayscale image, a region whose grayscale value is less than a preset grayscale threshold as the effective pathological region.

In an embodiment, the content evaluation module includes:
an area determining unit, configured to determine a first area of the tumor cell region, and determine a second area of the effective pathological region; and
a content evaluation unit, configured to calculate a tumor proportion and a tumor-stroma ratio of the digital pathology slide image based on the first area and the second area.

In an embodiment, the tumor cell content evaluation system further includes:
a diameter obtaining module, configured to obtain a plurality of test diameters corresponding to a plurality of tumor cells, and calculate a mean value of the plurality of test diameters, to obtain an average diameter of a single tumor cell;
an area calculation module, configured to determine an average area of the single tumor cell based on the average diameter of the single tumor cell;
a cell quantity calculation module, configured to calculate a quantity of tumor cells per unit area based on the average area of the single tumor cell; and
a first quantity calculation module, configured to calculate a first quantity of tumor cells based on the first area and the quantity of tumor cells per unit area.

In an embodiment, the tumor cell content evaluation system further includes: a second quantity determining module, configured to perform cell segmentation on the tumor cell region by using a cell segmentation algorithm, to determine a second quantity of tumor cells.

In an embodiment, the tumor cell content evaluation system further includes:
a training sample obtaining module, configured to obtain a training sample set, where the training sample set includes a training pathological region and a corresponding training cell type; and
a classifier training module, configured to use the training pathological region as an input of a preset classifier, use the training cell type as an expected output, and train the preset classifier, to obtain the pathology image classifier for which training is completed.

In an embodiment, the tumor cell content evaluation system further includes:
a test sample obtaining module, configured to obtain a test sample set, where the test sample set includes a test effective region and a corresponding test cell type;
a classification test module, configured to input the test effective region to the preset classifier, to obtain an output verification cell type; and
a verification module, configured to: obtain an error between the verification cell type and the test cell type, and when the error is less than a preset error, determine that training for the preset classifier is completed; or
obtain a quantity of training times corresponding to the preset classifier, and when the quantity of training times reaches a maximum preset quantity, determine that training for the preset classifier is completed.

FIG. 9 is a diagram of an internal structure of a computer device according to an embodiment. The computer device may be specifically a server, and the server includes but is not limited to a high-performance computer or a high-performance computer cluster. As shown in FIG. 9, the computer device includes a processor, a memory, and a network interface that are connected by using a system bus. The memory includes a non-volatile storage medium and an internal memory. The non-volatile storage medium of the computer device stores an operating system, and may further store computer-readable instructions. When the computer-readable instructions are executed by the processor, the processor may be enabled to implement a tumor cell content evaluation method. The internal memory may also store computer-readable instructions. When the computer-readable instructions are executed by the processor, the processor may be enabled to perform the tumor cell content evaluation method. A person skilled in the art may understand that the structure shown in FIG. 9 is merely a block diagram of a partial structure related to the solutions of this application, and does not constitute a limitation on a computer device to which the solutions of this application are applied. A specific computer device may include more or fewer components than those shown in the figure, or combine some components, or have different component arrangements.

In an embodiment, the tumor cell content evaluation method provided in this application may be implemented in a form of computer-readable instructions, and the computer-readable instructions may run on the computer device shown in FIG. 9. The memory of the computer device may store various program modules constituting the tumor cell content evaluation system, for example, the region determining module 802, the tumor cell identification module 804, and the content evaluation module 806.

A computer device is provided, including a memory, a processor, and computer-readable instructions stored in the memory and capable of running on the processor. When the processor executes the computer-readable instructions, the following steps are implemented: obtaining a digital pathology slide image, and determining an effective pathological region based on the digital pathology slide image; identifying a tumor cell region corresponding to the effective pathological region by using a deep learning-based pathology image classifier; and determining tumor cell content of the digital pathology slide image based on the tumor cell region according to a preset evaluation rule.

In an embodiment, the determining an effective pathological region based on the digital pathology slide image includes: performing binarization processing on the digital pathology slide image to obtain a grayscale image; and extracting, from the grayscale image, a region whose grayscale value is less than a preset grayscale threshold as the effective pathological region.

In an embodiment, the determining tumor cell content of the digital pathology slide image based on the tumor cell region according to a preset evaluation rule includes: determining a first area of the tumor cell region, and determining a second area of the effective pathological region; and calculating a tumor proportion and a tumor-stroma ratio of the digital pathology slide image based on the first area and the second area.

In an embodiment, after the determining tumor cell content of the digital pathology slide image based on the tumor cell region according to a preset evaluation rule, the method further includes: obtaining a plurality of test diameters corresponding to a plurality of tumor cells, and calculating a mean value of the plurality of test diameters, to obtain an average diameter of a single tumor cell; determining an average area of the single tumor cell based on the average diameter of the single tumor cell; calculating a quantity of tumor cells per unit area based on the average area of the single tumor cell; and calculating a first quantity of tumor cells based on the first area and the quantity of tumor cells per unit area.

In an embodiment, after the determining tumor cell content of the digital pathology slide image based on the tumor cell region according to a preset evaluation rule, the method further includes: performing cell segmentation on the tumor cell region by using a cell segmentation algorithm, to determine a second quantity of tumor cells.

In an embodiment, the tumor cell content evaluation method further includes: obtaining a training sample set, where the training sample set includes a training pathological region and a corresponding training cell type; and using the training pathological region as an input of a preset classifier, using the training cell type as an expected output, and training the preset classifier, to obtain the pathology image classifier for which training is completed.

In an embodiment, before the using the training pathological region as an input of a preset classifier, using the training cell type as an expected output, and training the preset classifier, to obtain the pathology image classifier for which training is completed, the method further includes: obtaining a test sample set, where the test sample set includes a test effective region and a corresponding test cell type; inputting the test effective region to the preset classifier, to obtain an output verification cell type; and obtaining an error between the verification cell type and the test cell type, and when the error is less than a preset error, determining that training for the preset classifier is completed; or obtaining a quantity of training times corresponding to the preset classifier, and when the quantity of training times reaches a maximum preset quantity, determining that training for the preset classifier is completed.

One or more non-volatile readable storage media storing computer-readable instructions are provided. In the one or more non-volatile readable storage media storing the computer-readable instructions, when the computer-readable instructions are executed by one or more processors, the one or more processors are enabled to perform the following steps: obtaining a digital pathology slide image, and determining an effective pathological region based on the digital pathology slide image; identifying a tumor cell region corresponding to the effective pathological region by using a deep learning-based pathology image classifier; and determining tumor cell content of the digital pathology slide image based on the tumor cell region according to a preset evaluation rule.

In an embodiment, the determining an effective pathological region based on the digital pathology slide image includes: performing binarization processing on the digital pathology slide image to obtain a grayscale image; and extracting, from the grayscale image, a region whose grayscale value is less than a preset grayscale threshold as the effective pathological region.

In an embodiment, the determining tumor cell content of the digital pathology slide image based on the tumor cell region according to a preset evaluation rule includes: determining a first area of the tumor cell region, and determining a second area of the effective pathological region; and calculating a tumor proportion and a tumor-stroma ratio of the digital pathology slide image based on the first area and the second area.

In an embodiment, after the determining tumor cell content of the digital pathology slide image based on the tumor cell region according to a preset evaluation rule, the method further includes: obtaining a plurality of test diameters corresponding to a plurality of tumor cells, and calculating a mean value of the plurality of test diameters, to obtain an average diameter of a single tumor cell; determining an average area of the single tumor cell based on the average diameter of the single tumor cell; calculating a quantity of tumor cells per unit area based on the average area of the single tumor cell; and calculating a first quantity of tumor cells based on the first area and the quantity of tumor cells per unit area.

In an embodiment, after the determining tumor cell content of the digital pathology slide image based on the tumor cell region according to a preset evaluation rule, the method further includes: performing cell segmentation on the tumor cell region by using a cell segmentation algorithm, to determine a second quantity of tumor cells.

In an embodiment, the tumor cell content evaluation method further includes: obtaining a training sample set, where the training sample set includes a training pathological region and a corresponding training cell type; and using the training pathological region as an input of a preset classifier, using the training cell type as an expected output, and training the preset classifier, to obtain the pathology image classifier for which training is completed.

In an embodiment, before the using the training pathological region as an input of a preset classifier, using the training cell type as an expected output, and training the preset classifier, to obtain the pathology image classifier for which training is completed, the method further includes: obtaining a test sample set, where the test sample set includes a test effective region and a corresponding test cell type; inputting the test effective region to the preset classifier, to obtain an output verification cell type; and obtaining an error between the verification cell type and the test cell type, and when the error is less than a preset error, determining that training for the preset classifier is completed; or obtaining a quantity of training times corresponding to the preset classifier, and when the quantity of training times reaches a maximum preset quantity, determining that training for the preset classifier is completed.

A person of ordinary skill in the art may understand that all or some of the processes in the methods in the foregoing embodiments may be implemented by computer-readable instructions instructing related hardware. The program may be stored in a non-volatile computer-readable storage medium. When the program is executed, the processes in the foregoing method embodiments may be included. Any reference to a memory, a storage, a database, or another medium used in the embodiments provided in this application may include a non-volatile and/or volatile memory. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory. As an illustration but not a limitation, the RAM may be obtained in a plurality of forms, such as a static RAM (SRAM), a dynamic RAM (DRAM), a synchronous DRAM (SDRAM), a double data rate SDRAM (DDRSDRAM), an enhanced SDRAM (ESDRAM), a synchronous link (Synchlink) DRAM (SLDRAM), a Rambus (Rambus) direct RAM (RDRAM), a direct Rambus dynamic RAM (DRDRAM), and a Rambus dynamic RAM (RDRAM).

The technical features in the foregoing embodiments may be combined randomly. For brief description, not all possible combinations of the technical features in the foregoing embodiments are described. However, the combinations of the technical features should be considered to fall within the scope described in this specification, provided that there is no contradiction between the combinations of the technical features.

The foregoing embodiments represent only several implementations of this application, and descriptions thereof are relatively specific and detailed, but may not be construed as a limitation on the scope of this application. It should be noted that a person of ordinary skill in the art may further make some modifications and improvements without departing from the concept of this application, and the modifications and improvements all fall within the protection scope of this application. Therefore, the protection scope of this application shall be subject to the appended claims.

1. A tumor cell content evaluation method, comprising:
obtaining a digital pathology slide image, and determining an effective pathological region based on the digital pathology slide image;
identifying, by using a deep learning-based pathology image classifier, a tumor cell region corresponding to the effective pathological region; and
determining tumor cell content of the digital pathology slide image based on the tumor cell region according to a preset evaluation rule.

2. The tumor cell content evaluation method according to claim 1, wherein the determining an effective pathological region based on the digital pathology slide image comprises:
performing binarization processing on the digital pathology slide image to obtain a grayscale image; and
extracting, from the grayscale image, a region whose grayscale value is less than a preset grayscale threshold as the effective pathological region.

3. The tumor cell content evaluation method according to claim 1, wherein the determining tumor cell content of the digital pathology slide image based on the tumor cell region according to a preset evaluation rule comprises:
determining a first area of the tumor cell region, and determining a second area of the effective pathological region; and
calculating a tumor proportion and a tumor-stroma ratio of the digital pathology slide image based on the first area and the second area.

4. The tumor cell content evaluation method according to claim 3, wherein after the determining tumor cell content of the digital pathology slide image based on the tumor cell region according to a preset evaluation rule, the method further comprises:
obtaining a plurality of test diameters corresponding to a plurality of tumor cells, and calculating a mean value of the plurality of test diameters, to obtain an average diameter of a single tumor cell;
determining an average area of the single tumor cell based on the average diameter of the single tumor cell;
calculating a quantity of tumor cells per unit area based on the average area of the single tumor cell; and
calculating a first quantity of tumor cells based on the first area and the quantity of tumor cells per unit area.

5. The tumor cell content evaluation method according to claim 1, wherein after the determining tumor cell content of the digital pathology slide image based on the tumor cell region according to a preset evaluation rule, the method further comprises:
performing cell segmentation on the tumor cell region by using a cell segmentation algorithm, to determine a second quantity of tumor cells.

6. The tumor cell content evaluation method according to claim 1, wherein the method further comprises:
obtaining a training sample set, wherein the training sample set comprises a training pathological region and a corresponding training cell type; and
using the training pathological region as an input of a preset classifier, using the training cell type as an expected output, and training the preset classifier, to obtain the pathology image classifier for which training is completed.

7. The tumor cell content evaluation method according to claim 6, wherein before the using the training pathological region as an input of a preset classifier, using the training cell type as an expected output, and training the preset classifier, to obtain the pathology image classifier for which training is completed, the method further comprises:
obtaining a test sample set, wherein the test sample set comprises a test effective region and a corresponding test cell type;
inputting the test effective region to the preset classifier, to obtain an output verification cell type; and
obtaining an error between the verification cell type and the test cell type, and when the error is less than a preset error, determining that training for the preset classifier is completed; or
obtaining a quantity of training times corresponding to the preset classifier, and when the quantity of training times reaches a maximum preset quantity, determining that training for the preset classifier is completed.

8. A tumor cell content evaluation system, wherein the tumor cell content evaluation system comprises:
a region determining module, configured to obtain a digital pathology slide image, and determine an effective pathological region based on the digital pathology slide image;
a tumor cell identification module, configured to identify, by using a deep learning-based pathology image classifier, a tumor cell region corresponding to the effective pathological region; and
a content evaluation module, configured to determine tumor cell content of the digital pathology slide image based on the tumor cell region according to a preset evaluation rule.

9. The tumor cell content evaluation system according to claim 8, wherein the region determining module comprises:
a binarization processing unit, configured to perform binarization processing on the digital pathology slide image to obtain a grayscale image; and
a region determining unit, configured to extract, from the grayscale image, a region whose grayscale value is less than a preset grayscale threshold as the effective pathological region.

10. The tumor cell content evaluation system according to claim 8, wherein the content evaluation module comprises:
an area determining unit, configured to determine a first area of the tumor cell region, and determine a second area of the effective pathological region; and
a content evaluation unit, configured to calculate a tumor proportion and a tumor-stroma ratio of the digital pathology slide image based on the first area and the second area.

11. The tumor cell content evaluation system according to claim 10, wherein the tumor cell content evaluation system further comprises:
a diameter obtaining module, configured to obtain a plurality of test diameters corresponding to a plurality of tumor cells, and calculate a mean value of the plurality of test diameters, to obtain an average diameter of a single tumor cell;
an area calculation module, configured to determine an average area of the single tumor cell based on the average diameter of the single tumor cell;
a cell quantity calculation module, configured to calculate a quantity of tumor cells per unit area based on the average area of the single tumor cell; and
a first quantity calculation module, configured to calculate a first quantity of tumor cells based on the first area and the quantity of tumor cells per unit area.

12. The tumor cell content evaluation system according to claim 8, wherein the tumor cell content evaluation system further comprises:
a second quantity determining module, configured to perform cell segmentation on the tumor cell region by using a cell segmentation algorithm, to determine a second quantity of tumor cells.

13. The tumor cell content evaluation system according to claim 8, wherein the tumor cell content evaluation system further comprises:
a training sample obtaining module, configured to obtain a training sample set, wherein the training sample set comprises a training pathological region and a corresponding training cell type; and
a classifier training module, configured to use the training pathological region as an input of a preset classifier, use the training cell type as an expected output, and train the preset classifier, to obtain the pathology image classifier for which training is completed.

14. The tumor cell content evaluation system according to claim 13, wherein the tumor cell content evaluation system further comprises:
a test sample obtaining module, configured to obtain a test sample set, wherein the test sample set comprises a test effective region and a corresponding test cell type;
a classification test module, configured to input the test effective region to the preset classifier, to obtain an output verification cell type; and
a verification module, configured to: obtain an error between the verification cell type and the test cell type, and when the error is less than a preset error, determine that training for the preset classifier is completed; or
obtain a quantity of training times corresponding to the preset classifier, and when the quantity of training times reaches a maximum preset quantity, determine that training for the preset classifier is completed.

15. A computer device, comprising a memory, a processor, and computer-readable instructions stored in the memory and capable of running on the processor, wherein when the processor executes the computer-readable instructions, the following steps are implemented:
obtaining a digital pathology slide image, and determining an effective pathological region based on the digital pathology slide image;
identifying, by using a deep learning-based pathology image classifier, a tumor cell region corresponding to the effective pathological region; and
determining tumor cell content of the digital pathology slide image based on the tumor cell region according to a preset evaluation rule.

16. The computer device according to claim 15, wherein the determining an effective pathological region based on the digital pathology slide image comprises:
performing binarization processing on the digital pathology slide image to obtain a grayscale image; and
extracting, from the grayscale image, a region whose grayscale value is less than a preset grayscale threshold as the effective pathological region.

17. The computer device according to claim 15, wherein the determining tumor cell content of the digital pathology slide image based on the tumor cell region according to a preset evaluation rule comprises:
determining a first area of the tumor cell region, and determining a second area of the effective pathological region; and
calculating a tumor proportion and a tumor-stroma ratio of the digital pathology slide image based on the first area and the second area.

18. The computer device according to claim 17, wherein after the determining tumor cell content of the digital pathology slide image based on the tumor cell region according to a preset evaluation rule, the method further comprises:
obtaining a plurality of test diameters corresponding to a plurality of tumor cells, and calculating a mean value of the plurality of test diameters, to obtain an average diameter of a single tumor cell;
determining an average area of the single tumor cell based on the average diameter of the single tumor cell;
calculating a quantity of tumor cells per unit area based on the average area of the single tumor cell; and
calculating a first quantity of tumor cells based on the first area and the quantity of tumor cells per unit area.

19. The computer device according to claim 15, wherein after the determining tumor cell content of the digital pathology slide image based on the tumor cell region according to a preset evaluation rule, the method further comprises:
performing cell segmentation on the tumor cell region by using a cell segmentation algorithm, to determine a second quantity of tumor cells.

20. The computer device according to claim 15, wherein when the processor executes the computer-readable instructions, the following steps are further implemented:
obtaining a training sample set, wherein the training sample set comprises a training pathological region and a corresponding training cell type; and
using the training pathological region as an input of a preset classifier, using the training cell type as an expected output, and training the preset classifier, to obtain the pathology image classifier for which training is completed.
